(19) United States
(12) Patent Application Publication
(10) Pub. No.: US 2013/0165799 A1
(43) Pub. Date: Jun. 27, 2013

(54) BIO-SIGNAL TRANSFER DEVICE, BIO-SIGNAL MONITORING SYSTEM, AND METHOD USING THE SAME

(71) Applicants: YongWon JANG, Daejeon (KR); Hyung Wook NOH, Daejeon (KR); Inbum LEE, Daejeon (KR); Yoon Seon SONG, Daejeon (KR); Sooyeul LEE, Daejeon (KR); Seunghwan KIM, Daejeon (KR)

(72) Inventors: YongWon JANG, Daejeon (KR); Hyung Wook NOH, Daejeon (KR); Inbum LEE, Daejeon (KR); Yoon Seon SONG, Daejeon (KR); Sooyeul LEE, Daejeon (KR); Seunghwan KIM, Daejeon (KR)

(73) Assignee: Electronics and Telecommunications Research Institute, Daejeon (KR)

(21) Appl. No.: 13/620,480

(22) Filed: Sep. 14, 2012

(30) Foreign Application Priority Data
Dec. 21, 2011 (KR) 10-2011-0139310

(51) Int. Cl. A61B 5/0205; A61B 5/01; A61B 5/00

(52) U.S. Cl. 600/484; 600/300

(57) ABSTRACT

Disclosed is a bio-signal monitoring system which includes a bio-signal transfer device which measures and analyzes a bio-signal and sends the analyzed bio-signal and first identification information; a reception device which includes at least one or more receivers, each receiver transferring information received from the bio-signal transfer device and second identification information; and a monitoring server which analyzes information received from each receiver and judges location and physical condition of a user, wherein the first identification information includes ID of the user and ID of the bio-signal and the second identification information includes ID of the receiver.

### CROSS-REFERENCE TO RELATED APPLICATIONS

A claim for priority under 35 U.S.C. §119 is made to Korean Patent Application No. 10-2011-0139310 filed Dec. 21, 2011, in the Korean Intellectual Property Office, the entire contents of which are hereby incorporated by reference.

### BACKGROUND

The inventive concepts described herein relate to a bio-signal transfer device, a bio-signal monitoring system, and a method using the same.

A bio-signal measuring and analyzing system may be used to check a physical condition of a user and to recognize the emergency such as sudden cardiogenic shock, arrhythmia, and the like. For example, electro-cardiogram (ECG) may be used as a bio-signal to diagnose the emergency.

The cases that a wireless information transfer technique is applied to a medical field may increase along with development of the communication technology. In a bio-signal monitoring system, information may be collected in a wireless transmission and reception manner to cope with a physical condition and the emergency checked through a bio-signal. However, a bio-signal measured in real time may have a large information amount. Thus, a large transfer amount may be required to transfer information in a wireless manner. This may cause overload easily. Also, in the event that communication is made in a Bluetooth manner, hindrance may be generated due to interference at a dense space of users. This may make it difficult to collect information.

### SUMMARY

Example embodiments of the inventive concept provide a bio-signal monitoring system which comprises a bio-signal transfer device which measures and analyzes a bio-signal and sends the analyzed bio-signal and first identification information; a reception device which includes at least one or more receivers, each receiver transferring information received from the bio-signal transfer device and second identification information; and a monitoring server which analyzes information received from each receiver and judges location and physical condition of a user, wherein the first identification information includes ID of the user and ID of the bio-signal and the second identification information includes ID of the receiver.

In example embodiments, the ID of the bio-signal includes a transfer time of the bio-signal.

In example embodiments, the bio-signal transfer device comprises at least one electrode which is attached to a body of the user to measure a first bio-signal; a bio-signal processing module which is attached to a body of the user to measure a second bio-signal, generates a bio analysis signal by analyzing the first bio-signal and the second bio-signal, and sends the bio analysis signal and the first identification signal; and a lead line which connects the electrode and the bio-signal processing module.

In example embodiments, the bio analysis signal includes a breathing rate and whether arrhythmia is generated.

In example embodiments, the bio analysis signal further includes a body temperature.

In example embodiments, the bio analysis signal includes a danger signal associated with whether a body of the user is abnormal.

In example embodiments, the monitoring server outputs an emergence signal to the outside in response to the danger signal.

In example embodiments, the bio-signal processing module comprises a connector which is connected to the lead line to transfer the first bio-signal; a sensor which measures the second bio-signal; an analog signal processing unit which removes noise from the first and second bio-signals to amplify amplitudes of the first and second bio-signals; a digital signal processing unit which analyzes the bio-signal amplified by the analog signal processing unit; and an information transmission unit which sends the bio analysis signal analyzed by the digital signal processing unit and the first identification information.

In example embodiments, the analog signal processing unit comprises a signal collector which combines the first bio-signal and the second bio-signal; a filter which removes noise from a bio-signal combined by the signal collector; and an amplifier which amplifies the bio-signal provided from the filter.

In example embodiments, the signal collector combines the first and second bio-signals based on a difference between the first bio-signal and the second bio-signal.

In example embodiments, the digital signal processing unit comprises a microcontroller unit which converts a bio-signal amplified by the analog signal processing unit into a digital signal and analyzes the converted bio-signal; and a memory which stores an analyzed result and the converted bio-signal provided from the microcontroller unit.

In example embodiments, the bio-signal transfer device sends information discretely.

In example embodiments, the bio-signal transfer device sends information periodically.

In example embodiments, an information transfer period of the bio-signal transfer device is variable.

In example embodiments, the monitoring server compares the number of receivers sending the information with a limit value and adjusts the strength of a transmission signal of the bio-signal transfer device.

Example embodiments of the inventive concept also provide a bio-signal transfer device which comprises at least one electrode which is attached to a body of a user to measure a first bio-signal; and a bio-signal processing module which is attached to the body of the user to measure a second bio-signal, generates a bio analysis signal by analyzing the first and second bio-signals, and sends the bio analysis signal and the first identification information, wherein the first identification information includes ID of the user and ID of the bio-signal.

Example embodiments of the inventive concept also provide a monitoring method which comprises measuring and analyzing a bio-signal of a user; sending the analyzed bio-signal to at least one receiver with first identification information; sending information provided from the receiver and second identification information; adjusting the strength of a signal transferred to the receiver according to a result obtained by comparing the number of receivers sending the information with a limit value; and judging a physical condition and location information of the user by analyzing information provided from the receiver, wherein the first identification information includes ID of the user and ID of the bio-signal and the second identification information includes ID of the receiver.

In example embodiments, the monitoring method further comprises outputting an emergence signal to the outside when a body of the user is judged to be abnormal according to the physical condition of the user.

### BRIEF DESCRIPTION OF THE FIGURES

The above and other objects and features will become apparent from the following description with reference to the following figures, wherein like reference numerals refer to like parts throughout the various figures unless otherwise specified, and wherein

FIG. 1 is a diagram schematically illustrating a bio-signal monitoring system according to an embodiment of the inventive concept.

FIG. 2 is a detailed block diagram illustrating a bio-signal transfer device according to an embodiment of the inventive concept.

FIG. 3 is a diagram illustrating a bio-signal transfer device in FIG. 2.

FIG. 4 is a block diagram schematically illustrating a bio-signal processing module according to an embodiment of the inventive concept.

FIG. 5 is a block diagram schematically illustrating a reception device in FIG. 1 according to an embodiment of the inventive concept.

FIG. 6 is a diagram schematically illustrating a bio-signal monitoring system according to an embodiment of the inventive concept.

FIG. 7 is a flowchart illustrating a monitoring method according to an embodiment of the inventive concept.

### DETAILED DESCRIPTION

Embodiments will be described in detail with reference to the accompanying drawings. The inventive concept, however, may be embodied in various different forms, and should not be construed as being limited only to the illustrated embodiments. Rather, these embodiments are provided as examples so that this disclosure will be thorough and complete, and will fully convey the concept of the inventive concept to those skilled in the art. Accordingly, known processes, elements, and techniques are not described with respect to some of the embodiments of the inventive concept. Unless otherwise noted, like reference numerals denote like elements throughout the attached drawings and written description, and thus descriptions will not be repeated. In the drawings, the sizes and relative sizes of layers and regions may be exaggerated for clarity.

It will be understood that, although the terms “first”, “second”, “third”, etc., may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the inventive concept.

Spatially relative terms, such as “beneath”, “below”, “lower”, “under”, “above”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” or “under” other elements or features would then be oriented “above” the other elements or features. Thus, the exemplary terms “below” and “under” can encompass both an orientation of above and below. The device may be otherwise oriented (rotated 90 degrees or at other orientations) and the spatially relative descriptors used herein interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Also, the term “exemplary” is intended to refer to an example or illustration.

It will be understood that when an element or layer is referred to as being “on”, “connected to”, “coupled to”, or “adjacent to” another element or layer, it can be directly on, connected, coupled, or adjacent to the other element or layer, or intervening elements or layers may be present. In contrast, when an element is referred to as being “directly on,” “directly connected to”, “directly coupled to”, or “immediately adjacent to” another element or layer, there are no intervening elements or layers present.

Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and/or the present specification and will not be interpreted in an idealized or overly formal sense unless expressly so defined herein.

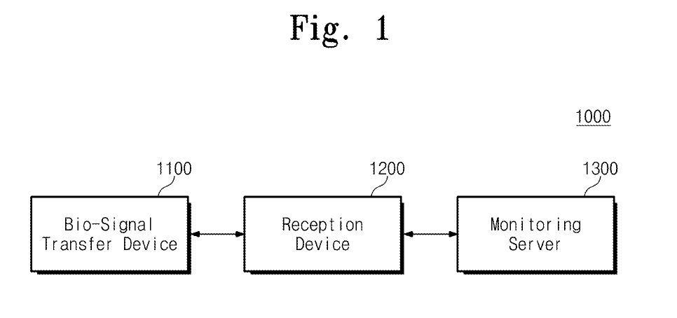

FIG. 1 is a diagram schematically illustrating a bio-signal monitoring system according to an embodiment of the inventive concept. Referring to FIG. 1, a bio-signal monitoring system 1000 may include a bio-signal transfer device 1100, a reception device 1200, and a monitoring server 1300.

The bio-signal transfer device 1100 may measure a bio-signal. The bio-signal transfer device 1100 may analyze the measured bio-signal. The bio-signal transfer device 1100 may add first identification information to the analyzed bio-signal to send it.

The reception device 1200 may receive information sent from the bio-signal transfer device 1100. The reception device 1200 may include at least one receiver. A receiver may be disposed within an appointed period. Each receiver receiving information may add second identification information to the received information to send it.

The monitoring server 1300 may combine and analyze information received from each reception device to comprehend physical condition and location information of a user.

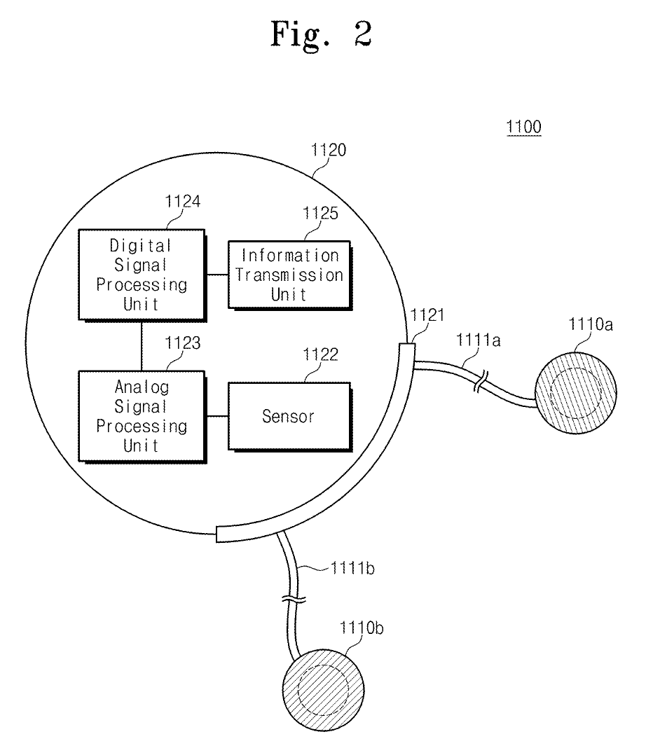

FIG. 2 is a detailed block diagram illustrating a bio-signal transfer device according to an embodiment of the inventive concept. Referring to FIG. 2, a bio-signal transfer device 1100 may include a first electrode 1110a, a first lead line 1111a, a second electrode 1110b, a second lead line 1111b, and a bio-signal processing module 1120.

The bio-signal transfer device 1100 may measure a bio-signal. In example embodiments, the bio-signal transfer device 1100 may measure a bio-signal generated by a physiological potential difference of a body of a user. The bio-signal measured by the bio-signal transfer device 1100 may be various. For example, the bio-signal may include electro-cardiogram (ECG), electro-encephalogram (EEG), electro-myogram (EMG), galvanic skin reflex (GSR), electro-oculography (EOG), Pulse plethysmography (PPG), amount of exercise on breathing rate and time, and the like. The inventive concept will be described on the basis of the electro-cardiogram (ECG). However, the inventive concept is not limited thereto. The bio-signal transfer device 1100 may analyze a measured bio-signal. The bio-signal transfer device 1100 may transfer the analyzed signal. The bio-signal transfer device 1100 may be attached to a user. In this case, the bio-signal transfer device 1100 may be a patch-type device.

The bio-signal transfer device 1100 may include at least one or more electrodes and lead lines. Each electrode may be connected to the bio-signal processing module 1120 through a lead line. In example embodiments, two electrodes 1110a and 1110b and two lead lines 1111a and 1111b may be used. However, the inventive concept is not limited thereto.

The first electrode 1110a may be connected to the bio-signal processing module 1120 through the first lead line 1111a. The second electrode 1110b may be connected to the bio-signal processing module 1120 through the second lead line 1111b. The first and second electrodes 1110a and 1110b may be a disposable electrode. The first and second electrodes 1110a and 1110b may be attached to a body of a user to measure a bio-signal.

The bio-signal processing module 1120 may measure a bio-signal. The bio-signal processing module 1120 may filter the measured bio-signal via a filter. The bio-signal processing module 1120 may analyze the filtered signal. The bio-signal processing module 1120 may send the analyzed signal. The bio-signal processing module 1120 may include a connector 1121, an analog signal processing unit 1123, a digital signal processing unit 1124, and an information transfer unit 1125.

The connector 1121 may be connected with at least one lead line. The connector 1121 may be connected to the bio-signal processing module 1120 through the lead line. In example embodiments, the connector 1121 may be connected to two lead lines 1111a and 1111b. However, the inventive concept is not limited thereto. For example, the connector 1121 may be connected with one lead line. Also, the connector 1121 can be connected with four or eight lead lines. A bio-signal measured by an electrode may be transferred to the connector 1121 through a lead line. The connector 1121 may transmit the input bio-signal to the analog signal processing unit 1123.

The sensor 1122 may measure a bio-signal. The sensor 1122 may be a disposable electrode. For example, the sensor 1122 may be configured the same as first and second electrodes. A bio-signal measured by the sensor 1122 may be various. For example, a bio-signal may include electro-cardiogram (ECG), electro-encephalogram (EEG), electro-myogram (EMG), galvanic skin reflex (GSR), electro-oculography (EOG), Pulse plethysmography (PPG), amount of exercise on breathing rate and time, and the like. The sensor 1122 may measure a bio-signal in various manners. For example, the sensor 1122 may measure a bio-signal generated due to a physiological potential difference of a body of a user. However, the inventive concept is not limited thereto. The sensor 1122 may send the measured bio-signal to the analog signal processing unit 1123.

The analog signal processing unit 1123 may remove noise of bio-signals measured by the sensor 1122, the first electrode 1110a, and the second electrode 1110b. The analog signal processing unit 1123 may amplify and output a noise-removed bio-signal.

The digital signal processing unit 1124 may receive a bio-signal amplified by the analog signal processing unit 1123. The digital signal processing unit 1124 may analyze a bio-signal to output an analyzed result. In example embodiments, the digital signal processing unit 1124 may analyze an electro-cardiogram signal to output whether or not arrhythmia is generated and its type. Alternatively, the digital signal processing unit 1124 may analyze an electro-cardiogram signal to output a breathing rate. Also, the digital signal processing unit 1124 may output a danger signal when an analyzed result of a bio-signal indicates that a body of a user is abnormal. Since the analyzed result has the information amount less than a raw bio-signal, it may be advantageous to transfer.

The information transfer unit 1125 may transfer the analyzed result of the digital signal processing unit 1124 in a wireless manner. The information transfer unit 1125 may add first identification information to the analyzed result of the digital signal processing unit 1124 to send it. The first identification information may include identification information of a user (hereinafter, referred to as user ID). The first identification information may include identification information of a currently transferred analyzed result (hereinafter, referred to as data ID). The data ID may be a time when a currently analyzed result is transferred.

Thus, the bio-signal transfer device 1100 according to an embodiment of the inventive concept may measure and analyze a bio-signal. A signal having the information amount reduced through analysis may be transmitted together with the first identification information by wireless.

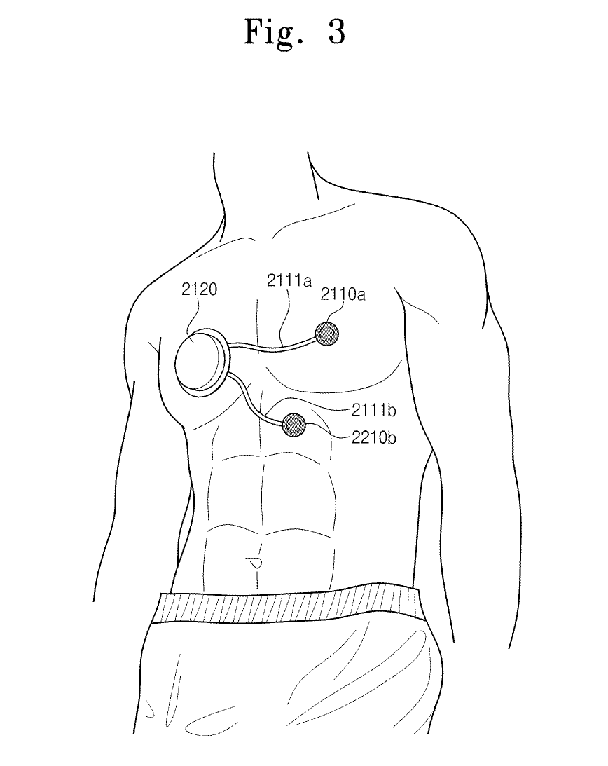

FIG. 3 is a diagram illustrating a bio-signal transfer device in FIG. 2. Referring to FIG. 3, a bio-signal transfer device may include a first electrode 2110a, a first lead line 2111a, a second electrode 2110b, a second lead line 2111b, and a bio-signal processing module 2120.

In example embodiments, the first electrode 2110a, the second electrode 2110b, and the bio-signal processing module 2120 may measure potentials of points corresponding to standard limb leads. The first electrode 2110a, the second electrode 2110b, and the bio-signal processing module 2120 may be attached to a body of a user. The first electrode 2110a may correspond to a left arm electrode, the second electrode 2110b to a left foot electrode, and the bio-signal processing module 2120 to a right arm electrode.

A potential difference between the bio-signal processing module 2120 and the first electrode 2110a may indicate a lead I signal. A potential difference between the bio-signal processing module 2120 and the second electrode 2110b may indicate a lead II signal. A potential difference between the first electrode 2110a and the second electrode 2110b may indicate a lead III signal. It is possible to measure a bio-signal easily using the lead signals. Bio-signals measured by the first electrode 2110a, the second electrode 2110b, and the bio-signal processing module 2120 may be processed and analyzed by the bio-signal module 2120. The analyzed information may be sent to the outside together with the first identification information.

Thus, the bio-signal transfer device according to an embodiment of the inventive concept may detect a bio-signal by measuring a potential difference between electrodes. The information amount of the detected bio-signal may be reduced through analysis, and then may be transferred to the outside.

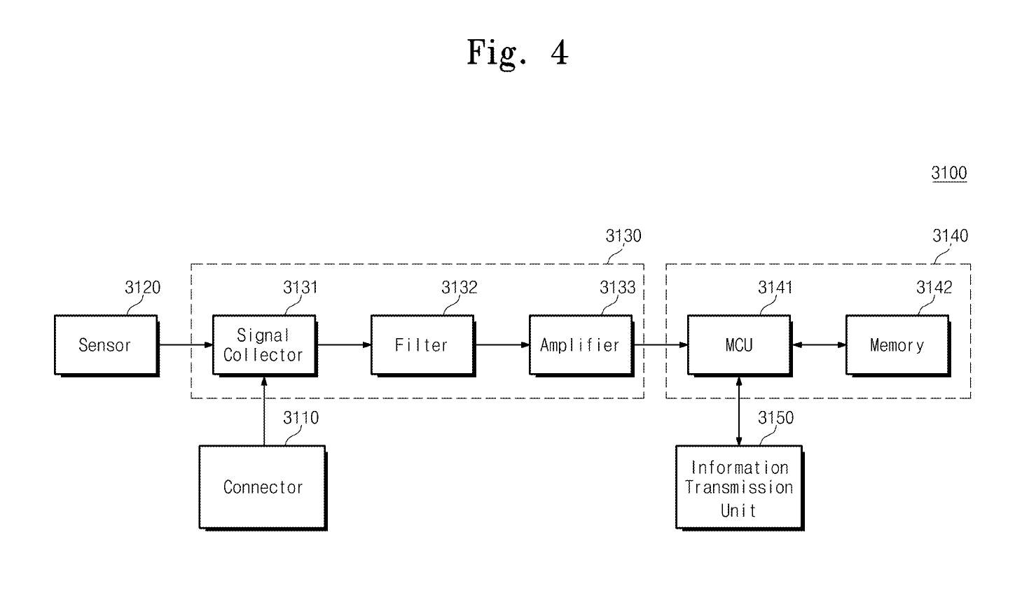

FIG. 4 is a block diagram schematically illustrating a bio-signal processing module according to an embodiment of the inventive concept. Referring to FIG. 4, a bio-signal processing module 3100 may include a connector 3110, a sensor 3120, an analog signal processing unit 3130, a digital signal processing unit 3140, and an information transfer unit 3150.

The connector 3110 may connect at least one lead line to the bio-signal processing module 3100. A bio-signal detected by an electrode may be transferred to the connector 3110 through a lead line. The connector 3110 may transfer the input bio-signal to the analog signal processing unit 3130.

The sensor 3120 may detect a bio-signal. The sensor 3120 may be attached to a body of a user. The sensor 3120 may measure a potential of an attached point.

The analog signal processing unit 3130 may include a signal collector 3131, a filter 3132, and an amplifier 3133. The analog signal processing unit 3130 may remove noise of bio-signals measured by the sensor 3120 and an electrode to amplify it.

The signal collector 3131 may receive a bio-signal measured by the sensor 3120. The signal collector 3131 may receive a bio-signal detected by an electrode through the connector 3110. The signal collector 3131 may combine bio-signals provided from the sensor 3120 and the connector 3110. The signal collector 3131 may send the combined information to the filter 3132. For example, the signal collector 3131 may provide lead I, II, and III signals to the filter 3132 based on a difference of potentials of respective points of a body input from the sensor 3120 and the connector 3110. If a potential difference between electrodes is used as a bio-signal, common mode noise may be removed. Thus, it is possible to obtain a signal with the high reliability.

The filter 3132 may remove noise of a bio-signal input from the signal collector 3131. The bio-signal may have a high noise property due to a small size. Also, since a body is organized by organic combination of organs, it is difficult to measure a signal associated with only a point. For example, a breathing signal may be measured together upon measuring of electro-cardiogram (ECG), or electro-myogram (EMG) may be measured together upon measuring of electro-encephalogram (EEG). Thus, a filter may be required to separate only a target signal from a measured signal. The filter 3132 may output a noise-removed bio-signal. In example embodiments, the filter 3132 may be placed between the signal collector 3131 and the amplifier 3133. However, the inventive concept is not limited thereto. For example, the filter 3132 may be placed following the amplifier 3133 to remove noise of an amplified signal.

The amplifier 3133 may amplify a bio-signal provided from the filter 3132. A magnitude of a bio-signal may be very small, for example, below 1 mV. Thus, amplification of a bio-signal may be required to analyze a bio-signal. The amplifier 3133 may output an amplified bio-signal.

The digital signal processing unit 3140 may include a microcontroller unit (MCU) 3141 and a memory 3142. The digital signal processing unit 3140 may convert a bio-signal provided from the analog signal processing unit 3130 into a digital signal to analyze the converted bio-signal.

The MCU 3141 may convert a bio-signal provided from the analog signal processing unit 3130 into a digital signal through an analog-to-digital converter. The MCU 3141 may include a digital filter. The digital filter may remove noise that is not removed by an analog filter.

The MCU 3141 may analyze a noise-removed bio-signal. In example embodiments, the MCU 3141 may analyze arrhythmia of a user and its type using the electro-cardiogram (ECG) signal. The MCU 3141 may analyze cardiogenic shock and cardiogenic disease of a user using the electro-cardiogram (ECG) signal. The MCU 3141 may store a bio-signal and an analyzed result at the memory 3142. Information stored at the memory 3142 may be used when a close analysis is required. The MCU 3141 may transfer the analyzed result to the information transfer unit 3150.

The information transfer unit 3150 may send the analyzed result input from the MCU 3141 by wireless. The information transfer unit 3150 may send first identification information together with the analyzed result. The first identification information may include user ID and data ID. The data ID may include a transfer time to identify a user associated with the currently transferred analyzed information. Also, since currently transferred analyzed information is sorted chronologically, it may be advantageous to analyze.

A bio-signal transfer device according to an embodiment of the inventive concept may be attached to a body to measure a bio-signal of a user in real time. The bio-signal transfer device may analyze a measured bio-signal to send only desired information. Thus, it is possible to reduce the amount of information to be transferred. Also, the bio-signal transfer device may perform a close analysis operation using identification information (e.g., time information of a bio-signal) as well as an analyzed bio-signal

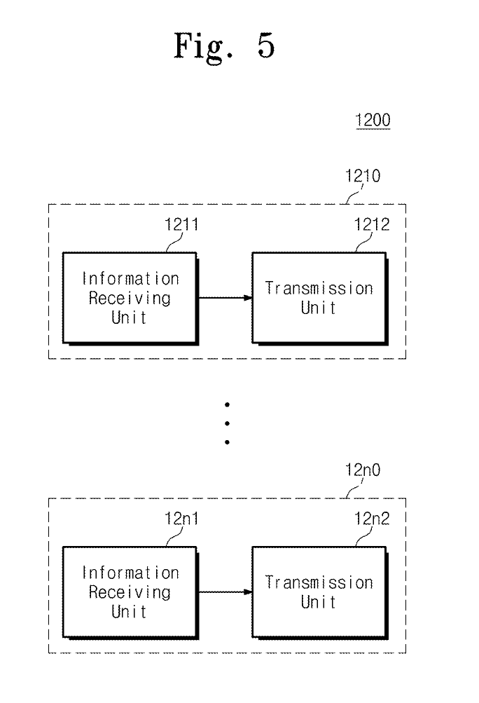

FIG. 5 is a block diagram schematically illustrating a reception device in FIG. 1 according to an embodiment of the inventive concept. Referring to FIG. 5, a reception device 1200 may include at least one or more receivers, each of which an information receiving unit and a transmission unit. Below, a reception device according to an embodiment of the inventive concept will be described using a first receiver 1210. The remaining receivers may be configured the same as the first receiver 1210.

The first receiver 1210 may receive information transferred from a bio-signal transfer device. The first receiver 1210 may send input information and second identification information to a monitoring server 1300.

An information receiving unit 1211 may receive information transferred from the bio-signal transfer device 1100. The information receiving unit 1211 may provide input information to a transmission unit 1212.

The transmission unit 1212 may send information transferred from the information receiving unit 1211 together with the second identification information to the monitoring server 1300. The second identification information may include an inherent ID of each receiver. This may make it possible for the monitoring server 1300 to comprehend a location of information received from the bio-signal transfer device 1100.

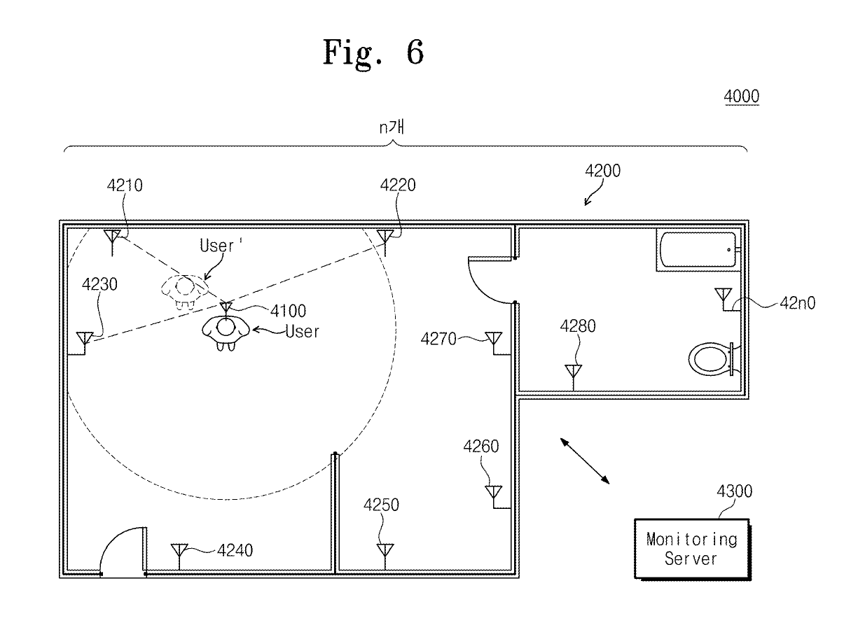

FIG. 6 is a diagram schematically illustrating a bio-signal monitoring system according to an embodiment of the inventive concept. Referring to FIG. 6, a bio-signal monitoring system 4000 may include a bio-signal transfer device 4100, a reception device 4200, and a monitoring server 4300. The reception device 4200 may include at least one receiver.

The bio-signal transfer device 4100 may be attached to a user. The bio-signal transfer device 4100 may measure a bio-signal of the user. The bio-signal transfer device 4100 may analyze the measured bio-signal. The bio-signal transfer device 4100 may transfer the analyzed bio-signal and first identification information.

The bio-signal transfer device 4100 may transfer information discretely or periodically. Compared with the case that information continues to be transferred, the bio-signal transfer device 4100 may be efficient in a power. Since information is transferred periodically, it may be possible to recognize omission of information easily. An information transfer period may be variable. For example, the information transfer period may be set to be long when the night where a location of a user is scarcely varied. Information transfer periods of different users may be set to be different from one another. When the monitoring server 4300 issues a command indicating reception of information, information may be instantly transferred regardless of an information transfer period.

An information transfer method of the bio-signal transfer device 4100 may not be limited to this disclosure. In example embodiments, the bio-signal transfer device 4100 may transfer information in a simple RF manner. In example embodiments, the bio-signal transfer device 4100 may transmit a less amount of information discretely. Thus, power consumption may be reduced by using a simple RF manner in which a complicated protocol is not used. However, the inventive concept is not limited thereto.

Information transferred from the bio-signal transfer device 4100 may be non-directional. Thus, information may be transmitted to a plurality of receivers within a transmission field. In example embodiments, it is assumed that information is transferred to first to third receivers 4210 to 4230.

The first to third receivers 4210 to 4230 may receive information from m the bio-signal transfer device 4100. The first to third receivers 4210 to 4230 may add second identification information to input information, respectively, to transfer resultant signals to the monitoring server 4300.

The monitoring server 4300 may receive information from the first to third receivers 4210 to 4230. The monitoring server 4300 may monitor a physical condition of a user using the input bio-signal information. The monitoring server 4300 may sort and classify bio-signal information using the first identification information.

The monitoring server 4300 may discriminate receives, which receive information from the bio-signal transfer device 4100, using the second identification information. The monitoring server 4300 may estimate a location of a user using locations of receivers receiving information from the bio-signal transfer device 4100.

In example embodiments, the monitoring server 4300 may use the estimated location of the user as an average location of receivers receiving information from the bio-signal transfer device 4100. However, the inventive concept is not limited thereto.

If a transmission field of the bio-signal transfer device 4100 is excessively wide, faraway receivers may receive signals. That is, the number of receivers receiving information may increase. Upon location estimation, an error may arise due to a distance difference between the bio-signal transfer device 4100 and receivers receiving information. If a transmission field of the bio-signal transfer device 4100 is excessively narrow, receivers close to the bio-signal transfer device 4100 may not receive signals. Thus, it is difficult to estimate a location.

To solve the above-described problems, the monitoring server 4300 may have upper limit and lower limit. If the number of receivers receiving information from the bio-signal transfer device 4100 is over the upper limit, the monitoring server 4300 may issue a command indicating a decrease in the strength of transmission. The bio-signal transfer device 4100 receiving the command may reduce a transmission field by lowering the strength of transmission signal. As the transmission field is reduced, the number of receivers receiving information from the bio-signal transfer device 4100 may decrease. Thus, it is possible to estimate a location of a user more exactly.

If the number of receivers receiving information from the bio-signal transfer device 4100 is below the lower limit, the monitoring server 4300 may issue a command indicating an increase in the strength of transmission. The bio-signal transfer device 4100 receiving the command may expand a transmission field by increasing the strength of transmission signal. As the transmission field is expanded, the number of receivers receiving information from the bio-signal transfer device 4100 may increase. Thus, it is possible to estimate a location of a user more exactly.

In the even that input bio-signal information includes a danger signal, the monitoring server 4300 may judge a body of the user to be abnormal. The monitoring server 4300 may send the estimated location and bio-signal information of the user to the outside, for example, a medical center to immediately cope with a sudden situation of a body of the user.

If no bio-signal information is received during a predetermined time, the monitoring server 4300 may issue a command indicating an increase in the strength of transmission. Nevertheless, if no bio-signal information is received during a predetermined time, the monitoring server 4300 may judge a body of the user to be abnormal. At this time, the monitoring server 4300 may output an emergency signal. The emergency signal may include warning through an output device such as alarm, image notification, and the like. Also, the emergency signal may include just recently estimated location and bio-signal information of the user. The monitoring server 4300 may send the emergency signal to the outside, for example, a medical center to immediately cope with a sudden situation of a body of the user.

The bio-signal monitoring system according to an embodiment of the inventive concept may be installed at various places such as a private residence, a hospital, a reception center, a nursing home, and the like. In this case, it is possible to analyze physical conditions and locations of many users.

The bio-signal monitoring system according to an embodiment of the inventive concept may continuously monitor physical condition and location information of a user. The bio-signal monitoring system may finely estimate location information of a user by adjusting the strength of signal. Also, the bio-signal monitoring system may immediately cope with a situation that a body of the user is abnormal, using danger signal and time information.

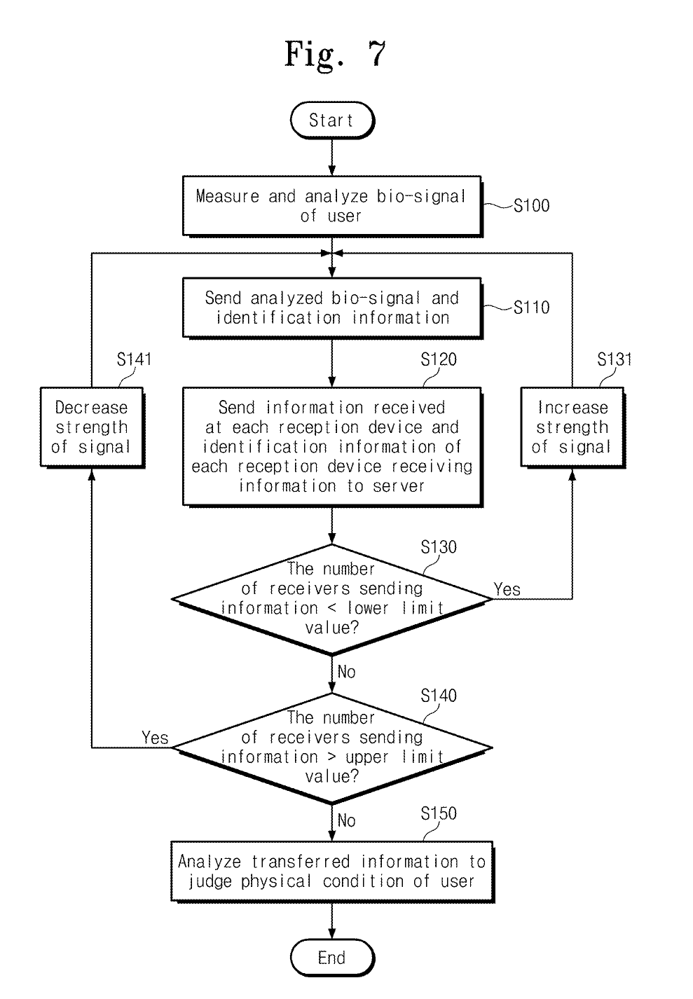

FIG. 7 is a flowchart illustrating a monitoring method according to an embodiment of the inventive concept. Referring to FIG. 7, in operation S100, a bio-signal of a user may be measured and analyzed. The bio-signal may include an electro-cardiogram (ECG) signal. A breathing rate, arrhythmia, a physical condition, and the like may be analyzed through the bio-signal. In operation S110, the analyzed bio-signal may be sent with first identification information, that is, user ID and data ID.

In operation S120, each reception device receiving information may send input information and own identification information to a monitoring server 1300. In operation S130, the monitoring server 1300 may compare the number of receivers sending information with a lower limit value. If the number of receivers sending information is below the lower limit value, in operation S131, the monitoring server 1300 may issue a command indicating an increase of the strength of a transmission signal.

In operation S140, the monitoring server 1300 may compare the number of receivers sending information with an upper limit value. If the number of receivers sending information is over the upper limit value, the monitoring server 1300 may issue a command indicating a decrease of the strength of a transmission signal.

When the number of receivers sending information is between the lower limit value and the upper limit value, in operation S150, the monitoring server 1300 may analyze input information to judge physical condition and location information of the user. If a body of the user is judged to be abnormal, the monitoring server 1300 may take a corresponding emergence action.

With the monitoring method according to an embodiment of the inventive concept, it is possible to continuously monitor physical condition and location information of a user. Also, it is possible to finely estimate location information of a user by adjusting the strength of signal. Accordingly, it is possible to immediately cope with a situation that a body of the user is abnormal, using danger signal and time information.

The inventive concept may be modified or changed variously. For example, a bio-signal transfer device 1100, a reception device 1200, and a monitoring server 1300 may be changed or modified variously according to environment and use.

While the inventive concept has been described with reference to exemplary embodiments, it will be apparent to those skilled in the art that various changes and modifications may be made without departing from the spirit and scope of the present invention. Therefore, it should be understood that the above embodiments are not limiting, but illustrative.

What is claimed is:

1. A bio-signal monitoring system comprising:
a bio-signal transfer device which measures and analyzes a bio-signal and sends the analyzed bio-signal and first identification information;
a reception device which includes at least one or more receivers, each receiver transferring information received from the bio-signal transfer device and second identification information; and
a monitoring server which analyzes information received from each receiver and judges location and physical condition of a user,
wherein the first identification information includes ID of the user and ID of the bio-signal and the second identification information includes ID of the receiver.

2. The bio-signal monitoring system of claim 1, wherein the ID of the bio-signal includes a transfer time of the bio-signal.

3. The bio-signal monitoring system of claim 1, wherein the bio-signal transfer device comprises:
at least one electrode which is attached to a body of the user to measure a first bio-signal;
a bio-signal processing module which is attached to a body of the user to measure a second bio-signal, generates a bio analysis signal by analyzing the first bio-signal and the second bio-signal, and sends the bio analysis signal and the first identification signal; and
a lead line which connects the electrode and the bio-signal processing module.

4. The bio-signal monitoring system of claim 3, wherein the bio analysis signal includes a breathing rate and whether arrhythmia is generated.

5. The bio-signal monitoring system of claim 4, wherein the bio analysis signal further includes a body temperature.

6. The bio-signal monitoring system of claim 3, wherein the bio analysis signal includes a danger signal associated with whether a body of the user is abnormal.

7. The bio-signal monitoring system of claim 6, wherein the monitoring server outputs an emergence signal to the outside in response to the danger signal.

8. The bio-signal monitoring system of claim 3, wherein the bio-signal processing module comprises:
a connector which is connected to the lead line to transfer the first bio-signal;
a sensor which measures the second bio-signal;
an analog signal processing unit which removes noise from the first and second bio-signals to amplify amplitudes of the first and second bio-signals;
a digital signal processing unit which analyzes the bio-signal amplified by the analog signal processing unit; and
an information transmission unit which sends the bio analysis signal analyzed by the digital signal processing unit and the first identification information.

9. The bio-signal monitoring system of claim 8, wherein the analog signal processing unit comprises:
a signal collector which combines the first bio-signal and the second bio-signal;
a filter which removes noise from a bio-signal combined by the signal collector; and
an amplifier which amplifies the bio-signal provided from the filter.

10. The bio-signal monitoring system of claim 9, wherein the signal collector combines the first and second bio-signals based on a difference between the first bio-signal and the second bio-signal.

11. The bio-signal monitoring system of claim 8, wherein the digital signal processing unit comprises:
a microcontroller unit which converts a bio-signal amplified by the analog signal processing unit into a digital signal and analyzes the converted bio-signal; and
a memory which stores an analyzed result and the converted bio-signal provided from the microcontroller unit.

12. The bio-signal monitoring system of claim 1, wherein the bio-signal transfer device sends information discretely.

13. The bio-signal monitoring system of claim 11, wherein the bio-signal transfer device sends information periodically.

14. The bio-signal monitoring system of claim 13, wherein an information transfer period of the bio-signal transfer device is variable.

15. The bio-signal monitoring system of claim 1, wherein the monitoring server compares the number of receivers sending the information with a limit value and adjusts the strength of a transmission signal of the bio-signal transfer device.

16. A bio-signal transfer device comprising:
at least one electrode which is attached to a body of a user to measure a first bio-signal; and
a bio-signal processing module which is attached to the body of the user to measure a second bio-signal, generates a bio analysis signal by analyzing the first and second bio-signals, and sends the bio analysis signal and the first identification information,
wherein the first identification information includes ID of the user and ID of the bio-signal.

17. A monitoring method comprising:
measuring and analyzing a bio-signal of a user;
sending the analyzed bio-signal to at least one receiver with first identification information;
sending information provided from the receiver and second identification information;
adjusting the strength of a signal transferred to the receiver according to a result obtained by comparing the number of receivers sending the information with a limit value; and
judging a physical condition and location information of the user by analyzing information provided from the receiver,
wherein the first identification information includes ID of the user and ID of the bio-signal and the second identification information includes ID of the receiver.

18. The monitoring method of claim 17, further comprising:
outputting an emergence signal to the outside when a body of the user is judged to be abnormal according to the physical condition of the user.
